(19) United States
(12) Patent Application Publication
(10) Pub. No.: US 2025/0072749 A1
(43) Pub. Date: Mar. 6, 2025

(54) MEASURING BIOMECHANICS IN REAL TIME AT MULTIPLE EYE-TISSUE LOCATIONS

(71) Applicant: Alcon Inc., Fribourg (CH)

(72) Inventors: Gangjun LIU, Portland, OR (US); Lingfeng YU, Rancho Santa Margarita, CA (US); Yilin ZHAO, Irvine, CA (US)

(21) Appl. No.: 18/816,959

(22) Filed: Aug. 27, 2024

Related U.S. Application Data
(60) Provisional application No. 63/579,406, filed on Aug. 29, 2023.

(51) Int. Cl. A61B 3/10; A61B 3/00; A61F 9/008

(52) U.S. Cl. CPC A61B 3/102; A61B 3/0041; A61F 2009/00897

(57) ABSTRACT

Certain aspects of the present disclosure provide systems and methods for measuring biomechanics in real-time at multiple eye-tissue locations. In certain embodiments, a method may be performed by a computer in communication with an optical coherence tomography (OCT) device. The method includes receiving an indication of a stimulus applied to eye tissue of a patient. The method also includes instructing the OCT device to emit a plurality of beams, at approximately the same time, to a plurality of measurement locations on the eye tissue in response to the received indication. The method also includes receiving, from the OCT device, OCT data for each of the plurality of measurement locations. The method also includes measuring tissue responses to the stimulus at the plurality of measurement locations based on the OCT data.

### CROSS-REFERENCE TO RELATED APPLICATIONS

This application claims priority to U.S. Provisional Application No. 63/579,406, filed on Aug. 29, 2023, which is hereby incorporated by reference in its entirety.

### INTRODUCTION

Tissue biomechanics, such as corneal biomechanics, can play an important role in understanding, diagnosing, and treating eye diseases such as glaucoma, Keratoconus, and ectasia. The ability to measure such biomechanics, however, is often restricted by hardware limitations. For example, it is often the case that optical coherence tomography (OCT) devices are not fast enough to capture useful measurements.

### SUMMARY

The present disclosure relates to diagnostic systems and methods, and more particularly, to systems and methods for measuring biomechanics in real-time at multiple eye-tissue locations.

In certain embodiments, one general aspect includes a system for measuring biomechanics in real-time at multiple eye-tissue locations. The system includes an optical coherence tomography (OCT) device and a computer communicably coupled to the OCT device. The computer is operable to receive an indication of a stimulus applied to eye tissue of a patient and, in response to the received indication, to instruct the OCT device to emit a plurality of beams, at approximately the same time, to a plurality of measurement locations on the eye tissue. The computer is also operable to receive, from the OC device, OCT data for each of the plurality of measurement locations. The computer is also operable to measure tissue responses to the stimulus at the plurality of measurement locations based on the OCT data.

In certain embodiments, another general aspect includes a method of measuring biomechanics in real-time at multiple eye-tissue locations. The method may be performed by a computer in communication with an optical coherence tomography (OCT) device. The method includes receiving an indication of a stimulus applied to eye tissue of a patient. The method also includes instructing the OCT device to emit a plurality of beams, at approximately the same time, to a plurality of measurement locations on the eye tissue in response to the received indication. The method also includes receiving, from the OCT device, OCT data for each of the plurality of measurement locations. The method also includes measuring tissue responses to the stimulus at the plurality of measurement locations based on the OCT data.

### BRIEF DESCRIPTION OF THE DRAWINGS

So that the manner in which the above-recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, and may admit to other equally effective embodiments.

FIG. 1A illustrates an example configuration of an ophthalmic diagnostics system, according to certain embodiments of the present disclosure.

FIG. 1B illustrates another example configuration of an ophthalmic diagnostics system, according to certain embodiments of the present disclosure.

FIG. 2 is a block diagram of various components of the ophthalmic diagnostics system of FIGS. 1A-B, according to certain embodiments of the present disclosure.

FIG. 3 illustrates example aspects of an ophthalmic diagnostics system, according to certain embodiments of the present disclosure.

FIG. 4 illustrates an example of an optical coherence tomography (OCT) engine operable to generate multiple imaging light beams, according to certain embodiments of the present disclosure.

FIG. 5 illustrates another example of an OCT engine operable to generate multiple imaging light beams, according to certain embodiments of the present disclosure.

FIG. 6 illustrates another example of an OCT engine operable to generate multiple imaging light beams, according to certain embodiments of the present disclosure.

FIGS. 7A-C illustrate example measurement patterns for an eye, according to certain embodiments of the present disclosure.

FIG. 8 illustrates an example of a process for measuring corneal biomechanics in real-time at multiple locations, according to certain embodiments of the present disclosure.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

For the purposes of promoting an understanding of the principles of the disclosure, reference will now be made to the implementations illustrated in the drawings, and specific language will be used to describe the same. It will nevertheless be understood that no limitation of the scope of the disclosure is intended. Any alterations and further modifications to the described systems, devices, instruments, methods, and any further application of the principles of the present disclosure are fully contemplated as would normally occur to one skilled in the art to which the disclosure relates In particular, the features, components, and/or steps described with respect to one implementation may be combined with the features, components, and/or steps described with respect to other implantations of the disclosure. For simplicity, in some instances the same reference numbers are used throughout the drawings to refer to the same or like parts.

Eye-tissue biomechanics, including corneal biomechanics such as corneal stiffness, can play an important role in understanding, diagnosing, and treating diseases such as glaucoma, Keratoconus, and ectasia. Detailed clinical assessment of corneal biomechanics has the potential to revolutionize the ophthalmic industry by providing, for example, personalized LASIK (Laser-Assisted in Situ Keratomileusis) and cataract surgery. Assessment and understanding of corneal biomechanics has application in corneal surgery for determining patient suitability and improving the safety and efficacy of surgery procedures. For example, corneal ectasia is a very rare but serious complication of refractive procedures that arises in 0.04-0.6% of cases. Measuring the corneal biomechanical properties can facilitate preoperative screening of refractive surgery candidates so as to minimize the risk of postoperative corneal ectasia. In cataract surgery, for example, surgically induced astigmatism (SIA) ranges from 0 to 1.5D can be a significant source of refractive surprises. Accurate measurement of corneal biomechanics can enable better prediction of patient-specific SIA for cataract surgery and increase the surgery outcomes.

One way to measure compressive stiffness, for example, is Young's modulus. Computation of Young's modulus typically involves measuring a speed of shear-wave propagation across a corneal surface in response to a stimulus. In theory, data generated by an optical coherence tomography (OCT) device can be used to measure the propagation speed. In practice, however, shear waves can propagate at speeds of tens of meters per second, for example. Generally, it only takes a few milliseconds for a shear wave to propagate the whole cornea. Since the speed of OCT devices is often less than 100 kilohertz, and OCT-based methods involve the scanning of laser beams, it is challenging to use OCT-based methods to measure the propagation speed of shear waves. Although it might be possible to avoid some of these technical difficulties by repeatedly applying a stimulus to the same location and then measuring the speed of shear-wave propagation at different locations at different times, such a process would be time-consuming and generally unsuitable for in-vivo applications.

The present disclosure describes examples of OCT-based methods for measuring biomechanics in real-time at multiple locations on a patient's eye. In various embodiments, a stimulus can be applied to the patient's cornea, for example. Thereafter, a computer can instruct, or cause, an OCT device to simultaneously emit multiple beams to multiple locations on the cornea. In various embodiments, the multiple beams result in OCT data being received for each of the multiple locations in response to a single stimulus. Advantageously, in certain embodiments, the OCT data can be used to measure corneal responses to the single stimulus at multiple locations, thereby greatly reducing measurement time. For example, a speed of shear-wave propagation can be more efficiently and reliably calculated based on OCT data simultaneously obtained from multiple locations. Further, in various examples, a speed of shear-wave propagation can be more easily measured in multiple directions. Particular examples will be described in greater detail relative to the Drawings.

For illustrative purposes, the present disclosure describes various examples in relation to measuring corneal biomechanics. However, it should be appreciated that similar principles are applicable to measuring biomechanics for other portions of the eye and/or other tissue.

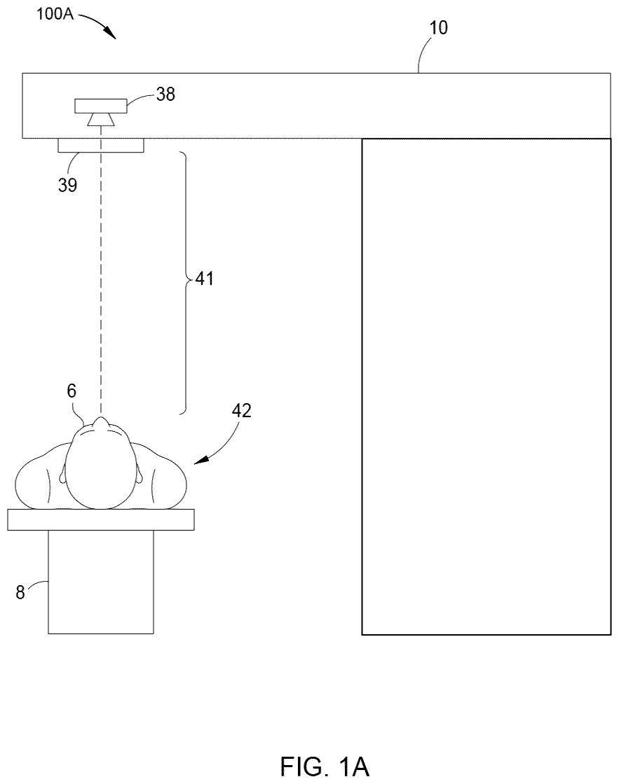

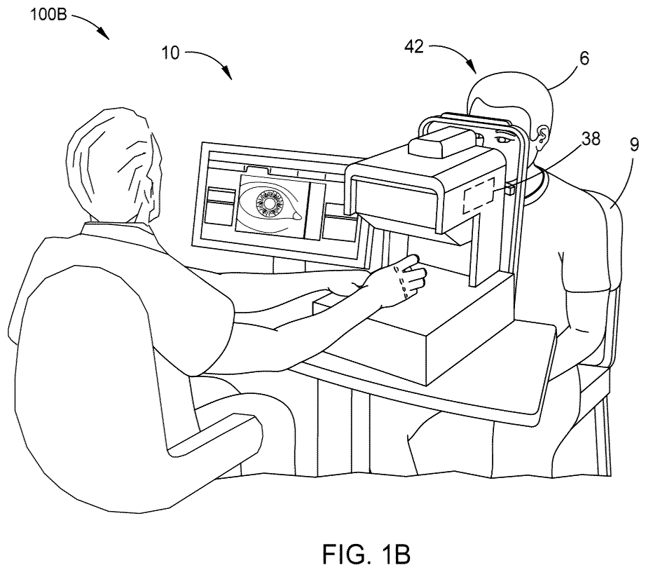

FIGS. 1A, 1B, and 2 illustrate an example of an ophthalmic diagnostics system 10 according to certain embodiments. The ophthalmic diagnostics system 10 may be used for different types of diagnostic and treatment procedures. For example, the ophthalmic diagnostics system 10 may be used for diagnosis or treatment of glaucoma, Keratoconus, and/or ectasia. In addition, or alternatively, the ophthalmic diagnostics system 10 may be used to provide data for personalizing LASIK or cataract surgery.

FIG. 1A shows a configuration 100A of the ophthalmic diagnostics system 10. In particular, FIG. 1A illustrates a head 6 of a patient 42 lying on a bed 8. In the illustrated example, the ophthalmic diagnostics system 10 includes a camera 38 and a part 39 where multiple imaging light beams can exit the ophthalmic diagnostics system 10 and travel through an area 41 towards the patient 42.

FIG. 1B shows a configuration 100B of the ophthalmic diagnostics system 10. In the configuration 100B, the ophthalmic diagnostics system 10 is configured as a desktop imaging system in which the patient 42 sits in a chair 9.

With reference to FIG. 2, the ophthalmic diagnostics system 10 includes an OCT device 15, the camera 38, and a control computer 30, coupled as shown. The OCT device 15 includes controllable components, such as an OCT engine 12, a beam scanner 16, one or more optical elements 17, and/or a focusing objective 18, coupled as shown. The computer 30 includes logic 36, a memory 32 (which stores a computer program 34), and a display 37, coupled as shown. For ease of explanation, the following xyz-coordinate system is used: The z-direction is defined by the propagation direction of the imaging light beams, and the xy-plane is orthogonal to the propagation direction. Other suitable xyz-coordinate systems may be used.

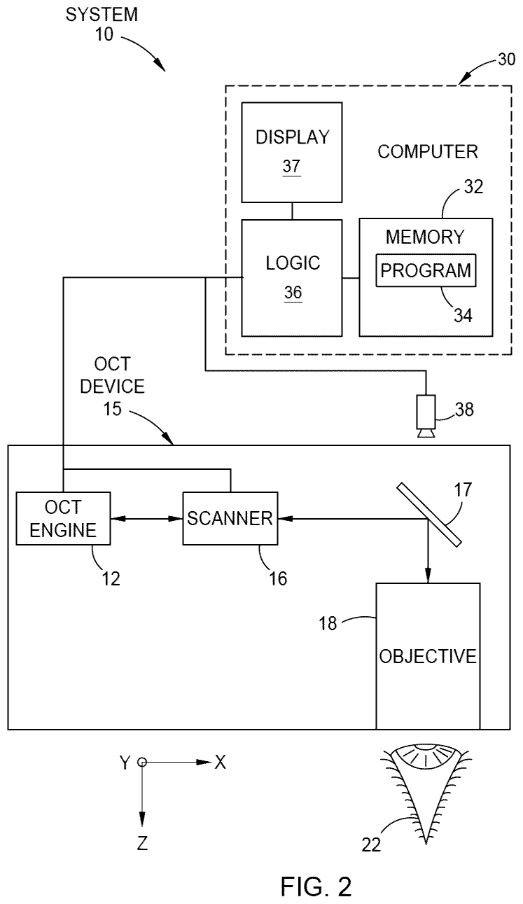

With particular reference to the OCT device 15, the OCT engine 12 generates and emits multiple imaging light beams that are guided to tissue of an eye 22 of the patient 42. For example, the imaging light beams may be guided to different locations on a corneal surface of the eye 22. As will be described in greater detail relative to FIGS. 3-6, 7A-C, and 8, the OCT engine 12 can generate and emit the imaging light beams at approximately the same time (i.e., simultaneously).

The beam scanner 16 variably guides the imaging light beams to the one or more optical elements 17 based on its configuration and positioning. For example, the beam scanner 16 can variably guide the imaging light beams by laterally and/or longitudinally directing the imaging light beams. The lateral direction refers to directions orthogonal to the direction of beam propagation, i.e., the x, y directions. The beam scanner 16 may laterally direct the imaging light beams in any suitable manner. For example, the beam scanner 16 may include a pair of galvanometrically-actuated scanner mirrors that can be tilted about mutually perpendicular axes. As another example, the beam scanner 16 may include an electro-optical crystal that can electro-optically steer the imaging light beams. In some embodiments, the beam scanner 16 can simultaneously adjust the imaging light beams emitted by the OCT engine 12.

The longitudinal direction refers to the direction parallel to the beam propagation, i.e., the z-direction. The beam scanner 16 may longitudinally direct the imaging light beams in any suitable manner. For example, the beam scanner 16 may include a longitudinally adjustable lens, a lens of variable refractive power, or a deformable mirror that can control the z-position of the beam focus. The components of the beam scanner 16 may be arranged in any suitable manner along applicable beam paths, e.g., in the same or different modular units.

The one or more optical elements 17 direct the imaging light beams towards the focusing objective 18. An optical element 17 can act on (e.g., transmit, reflect, refract, diffract, collimate, condition, shape, focus, modulate, and/or otherwise act on) the imaging light beams. Examples of optical elements include a lens, prism, mirror, diffractive optical element (DOE), holographic optical element (HOE), and spatial light modulator (SLM). In certain examples, the optical element 17 is a mirror or a dichroic mirror. The focusing objective 18 focuses the imaging light beams towards a portion of the eye 22, such as a corneal surface thereof. In the example, focusing objective 18 is an objective lens, e.g., an f-theta objective.

The OCT engine 12 receives returned imaging light beams, backscattered from the eye 22, along the opposite direction of the imaging light beams. The OCT engine 12 can be configured to generate an image or images to provide actionable feedback for storage, as described below in detail. For example, in various embodiments, the OCT engine 12 is configured to interferometrically analyze the returned imaging light beams to provide OCT data representing a position-dependent structural property of the eye 22, such as structural property of a cornea thereof. For example, the OCT engine 12 can be configured to provide OCT data representing an image of the cornea at or in the vicinity of the focal position x,y,z and to provide OCT data representing a position-dependent optical density n (x,y,z) of the cornea as well as a position-dependent mass density p (x,y,z) of the cornea.

Although certain examples of the OCT device 15 are described above, it should be appreciated that, in various embodiments, the OCT device 15 can be configured to conduct different types of OCT scans. In an example, in some embodiments, the OCT device 15 can be configured to execute a M-scan. In another example, the OCT device 15 can be configured to execute a B-scan. In yet another example, the OCT device 15 can be configured to execute an MB-scan. In still other examples, the OCT device 15 can be configured to execute a BM-scan. Other examples will be apparent to one skilled in the art after a detailed review of the present disclosure.

The camera 38 can continuously capture one or more images of the patient 42. For example, the camera 38 can be focused on the eye 22. Examples of the camera 38 include a video, interferometry, thermal imaging, ultrasound, OCT, and eye-tracking cameras. The camera 38 delivers image data, which represent recorded images of the eye 22, to the computer 30. In some embodiments, the camera 38 can be an integral part of the OCT device 15, rather than separate as illustrated in FIG. 2.

The computer 30 controls components of the ophthalmic diagnostics system 10 in accordance with the computer program 34. For example, the computer 30 controls components (e.g., the OCT engine 12, the beam scanner 16, the optical elements 17, and/or the focusing objective 18) to focus the imaging light beams of the OCT engine 12 at desired measurement locations on the eye 22, such as desired measurement locations on a corneal surface thereof. The memory 32 stores information used by the computer 30. For example, memory 32 may store images of the eye 22, OCT data, and/or other suitable information, and the computer 30 may access information from the memory 32. In various embodiments, the computer program 34 and its functionality, such as the focusing of the imaging light beams, can be directed by a user such as a medical professional.

In certain embodiments, the computer 30 can measure corneal biomechanics of the eye 22 in real-time at multiple locations. In certain embodiments, the computer 30 monitors for an indication of a stimulus applied to a patient's cornea. When the computer 30 detects a stimulus, or is notified of such a stimulus (e.g., by a user), the computer 30 can instruct, or cause, the OCT device 15 to emit multiple imaging light beams to multiple different locations on the patient's cornea. The computer 30 can then receive, from the OCT device 15, OCT data resultant from the imaging light beams. The OCT data can be used by the computer 30 to measure corneal responses to the stimulus at each of the multiple different locations in response to the detected stimulus. The computer 30 can record the OCT data and/or data related to the measured corneal responses in the memory 32 or other storage.

FIG. 3 illustrates an example of an ophthalmic diagnostics system 310. In general, the ophthalmic diagnostics system 310 can include any of the components and functionality described relative to the ophthalmic diagnostics system 10 of FIGS. 1A-B and 2. Similarly, the ophthalmic diagnostics system 10 can include any of the components and functionality described relative to the ophthalmic diagnostics system 310. Therefore, for ease of description, like components of the ophthalmic diagnostics system 10 and the ophthalmic diagnostics system 310 may be periodically referenced interchangeably. For simplicity, the illustration of FIG. 3 focuses on an example OCT device 315, an example camera 338, and a stimulation device 352.

In the illustrated embodiment, the OCT device 315 includes an OCT engine 312, a beam scanner 316, one or more optical elements 317, a focusing objective 318, and lights 348(1) and 348(2). In general, the OCT engine 312, the beam scanner 316, the one or more optical elements 317, and the focusing objective 318 can each operate as described relative to the OCT engine 12, the beam scanner 16, the one or more optical elements 17, and/or the focusing objective 18, respectively, of FIG. 2.

In similar fashion to the OCT engine 12 of FIG. 2, the OCT engine 312 generates and emits multiple imaging light beams that are guided to a surface of a cornea 346 of an eye 322, and can thereafter receive returned imaging light beams, backscattered from the eye 322, along the opposite direction of the imaging light beams. For illustrative purposes, the OCT engine 312 is shown as generating and emitting three imaging light beams, namely, imaging light beams 344(1), 344(2), and 344(2) (collectively, imaging light beams 344). It should be appreciated, however, that the quantity of the imaging light beams 344 is configurable to suit a given implementation.

In the example of FIG. 3, the beam scanner 316 variably guides the imaging light beams 344 to specific locations on the one or more optical elements 317, based on its configuration and/or positioning. The one or more optical elements 317 direct the imaging light beams 344 toward specific locations on the focusing objective 318, based on, or in accordance with, the specific locations thereon to which the imaging light beams 344 are guided by the beam scanner 316. For example, due to a position and/or location-varying properties of the one or more optical elements 317, different beams may be directed to different locations on the focusing objective 318. The focusing objective 318 can focus the imaging light beams 344 on a plurality of desired measurement locations on the cornea 346. In the example of FIG. 3, the one or more optical elements 317 are shown as a dichroic mirror, and the focusing objective 318 is shown as an objective lens.

In some embodiments, the camera 338 can be an iris camera that is focused or fixed on the eye 322, for example, and provides continuous images of the eye 322 to the computer 30. In various embodiments, the lights 348(1) and 348(2) can improve the quality of image capture by the camera 338. For illustrative purposes, the lights 348(1) and 348(2) are shown as light-emitting diodes (LEDs) that direct light toward the eye 322. The camera 338 can operate as described relative to the camera 38 of FIGS. 1A-B and 2. One skilled in the art will appreciate that the components shown in FIG. 3 can exist in any suitable number or configuration. For example, it should be appreciated that the two lights shown in FIG. 3, namely, lights 348(1) and 348(2), can be modified in quantity, type, and/or configuration to suit a given implementation.

The stimulation device 352 can be any suitable device for applying an external stimulus to the cornea 346. For example, the stimulation device 352 may apply an air pulse to induce a corneal displacement. In another example, the stimulation device 352 may apply an ultrasound wave to induce a corneal displacement. Other examples of stimulation will be apparent to one skilled in the art after a detailed review of the present disclosure.

In various embodiments, the computer 30 of FIG. 2 monitors for an indication of a stimulus applied to the cornea 346 by the stimulation device 352. When the computer 30 detects a stimulus applied by the stimulation device 352, or is notified of such as stimulus (e.g., by a user), the computer 30 can instruct, or cause, the OCT device 315 to emit the multiple imaging light beams 344, at approximately the same time (i.e., simultaneously), to multiple different locations on the cornea 346. The computer 30 can then receive, from the OCT device 315, OCT data resultant from the imaging light beams 344. The OCT data can be used by the computer 30 to measure corneal responses to the stimulus at each of the multiple different locations in response to the detected stimulus. The computer 30 can record the OCT data and/or data related to the measured corneal responses in the memory 32 or other storage.

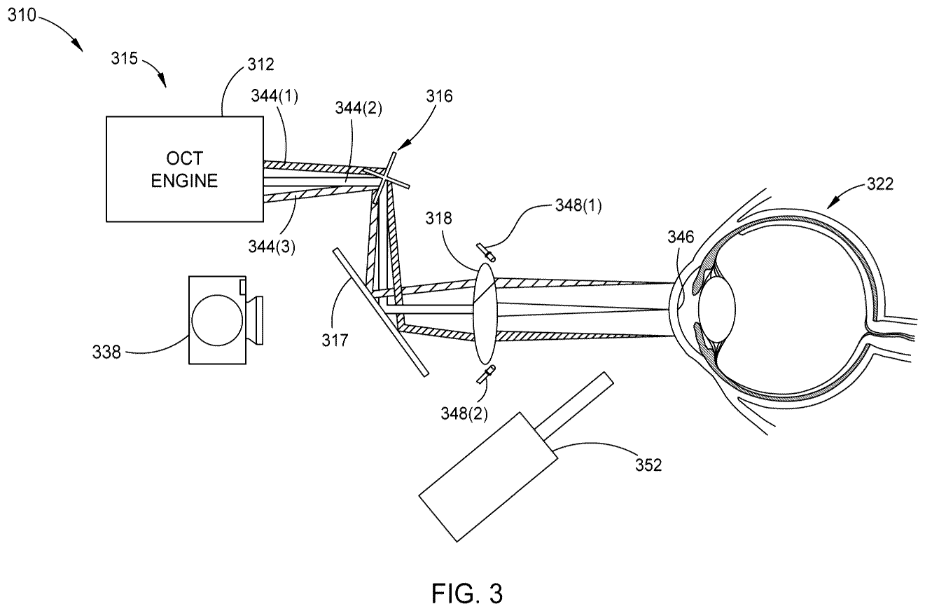

FIG. 4 illustrates an example of an OCT engine 412 operable to generate multiple imaging light beams. In various embodiments, the OCT engine 412 can serve as the OCT engine 12 of FIG. 2 and/or as the OCT engine 312 of FIG. 3. In the illustrated embodiment, the OCT engine 412 includes a beam source 454, a beam splitter 458, and optical elements 462(1) and 462(2).

In the illustrated embodiment, the beam source 454 produces a source beam 456 in any suitable fashion. The beam splitter 458 splits the source beam 456 into a first beam 460(1) to the optical element 462(1) and a second beam 460(2) to the optical element 462(2). The beam splitter 458 can be any suitable device for splitting light beams, such as a polarization beam splitter in some cases.

The optical elements 462(1) and 462(2) direct the beams 460(1) and 460(2), respectively, to the beam scanner 316, such that the beams 460(1) and 460(2) are the multiple imaging light beams generated by the OCT engine 412. The optical elements 462(1) and 462(2) can act on (e.g., transmit, reflect, refract, diffract, collimate, condition, shape, focus, modulate, and/or otherwise act on) the beams 460(1) and 460(2), respectively. Examples of the optical elements 462(1) and 462(2) include lenses, prisms, mirrors, diffractive optical elements (DOEs), holographic optical elements (HOEs), and spatial light modulators (SLM). In the example of FIG. 4, the optical elements 462(1) and 462(2) are mirrors. The beams 460(1) and 460(2) can be guided via the beam scanner 316 to a cornea, such as the cornea 346 of FIG. 3, as generally described relative to FIGS. 1A-B, 2 and 3.

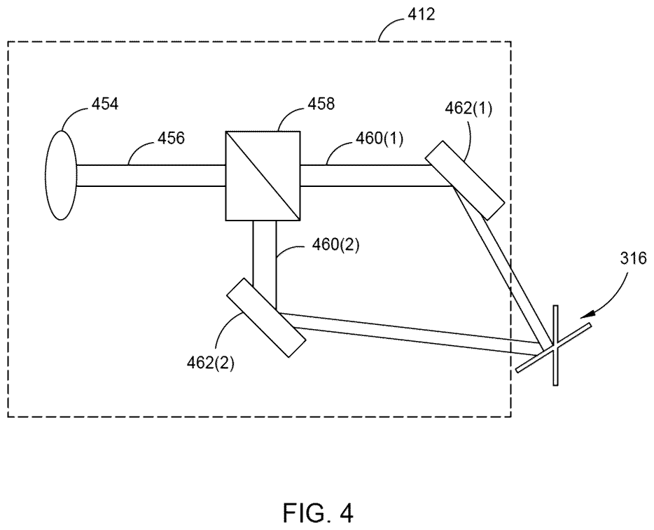

FIG. 5 illustrates an example of an OCT engine 512 operable to generate multiple imaging light beams. In various embodiments, the OCT engine 512 can serve as the OCT engine 12 of FIG. 2 and/or as the OCT engine 312 of FIG. 3. In the illustrated embodiment, the OCT engine 512 includes a beam source 554, a beam splitter 558, and optical elements 564(1), 564(2), 564(3), and 564(4) (collectively, optical elements 564).

In the illustrated embodiment, the beam source 554 produces a source beam 556 in any suitable fashion. The beam splitter 558 splits the source beam 556 into a first beam 560(1) directed to the optical element 564(1), a second beam 560(2) directed to the optical element 564(2), a third beam 560(3) directed to the optical element 564(3), and a fourth beam 560(4) directed to the optical element 564(4) (collectively, beams 560). The beam splitter 558 can be any suitable device for splitting light beams. In the example of FIG. 5, the beam splitter 558 is 1×N fiber beam splitter, with N equaling four in the illustrated embodiment. It should be appreciated, however, that the beam splitter 558 can split the source beam 556 into a different quantity of beams according to the requirements of a given implementation.

The optical elements 564 direct the beams 560 to the beam scanner 316, such that the beams 560 are the multiple imaging light beams generated by the OCT engine 512. The optical elements 564 can act on (e.g., transmit, reflect, refract, diffract, collimate, condition, shape, focus, modulate, and/or otherwise act on) the beams 560. Examples of the optical elements 564 include lenses, prisms, mirrors, diffractive optical elements (DOEs), holographic optical elements (HOEs), and spatial light modulators (SLM). In the example of FIG. 5, the optical elements 564 are collimators. The beams 560 can be guided via the beam scanner 316 to a cornea, such as the cornea 346 of FIG. 3, as generally described relative to FIGS. 1A-B, 2, and 3.

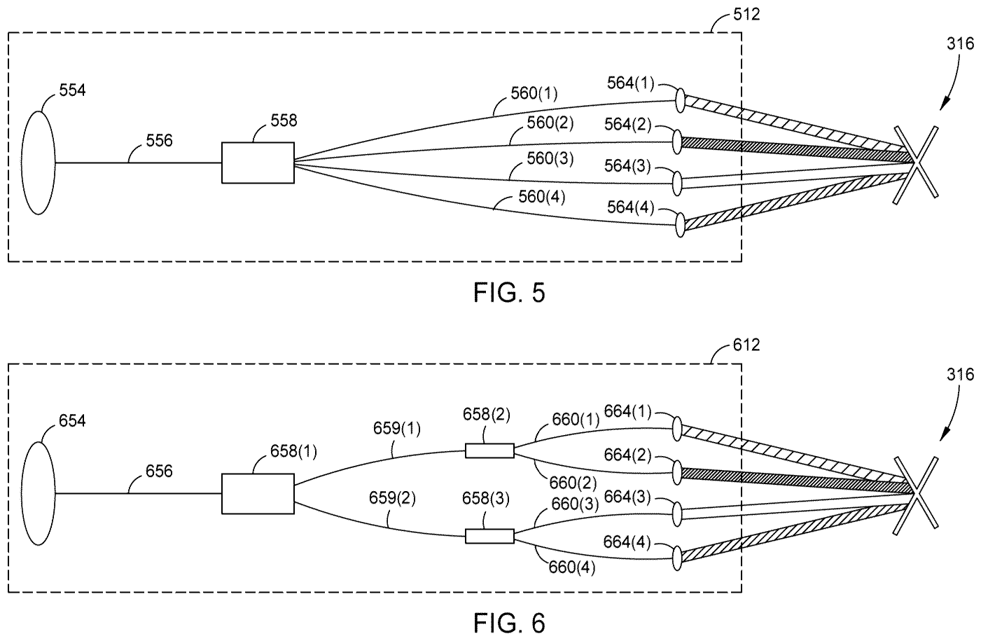

FIG. 6 illustrates an example of an OCT engine 612 operable to generate multiple imaging light beams. In various embodiments, the OCT engine 612 can serve as the OCT engine 12 of FIG. 2 and/or as the OCT engine 312 of FIG. 3. In the illustrated embodiment, the OCT engine 612 includes a beam source 654, beam splitters 658(1), 658(2) and 658(3), and optical elements 664(1), 664(2), 664(3), and 664(4) (collectively, optical elements 664). It should be appreciated that the beam splitters 658(1), 658(2), and 658(3) can be configured split light beams in any suitable fashion.

In the illustrated embodiment, the beam source 654 produces a source beam 656 in any suitable fashion. The beam splitter 658(1) splits the source beam 656 into a first intermediate beam 659(1) directed to the beam splitter 658(2) and a second intermediate beam 659(2) directed to the beam splitter 658(3). For clarity, the intermediate beams 659(1) and 659(2) are referred to as “intermediate” because they do not represent beams output by the OCT engine 612.

Continuing the example of FIG. 6, the beam splitter 658(2) splits the intermediate beam 659(1) into a first output beam 660(1) directed to the optical element 664(1) and a second output beam 660(2) directed to the optical element 664(2). Similarly, the beam splitter 658(3) splits the intermediate beam 659(2) into a third output beam 660(3) directed to the optical element 664(3) and a fourth output beam 660(4) directed to the optical element 664(4). Collectively, the output beams 660(1), 660(2), 660(3), and 660(4) will be referred to as output beams 660.

The optical elements 664 direct the output beams 660 to the beam scanner 316, such that the output beams 660 are the multiple imaging light beams generated by the OCT engine 612. The optical elements 664 can act on (e.g., transmit, reflect, refract, diffract, collimate, condition, shape, focus, modulate, and/or otherwise act on) the output beams 660. Examples of the optical elements 564 include lenses, prisms, mirrors, diffractive optical elements (DOEs), holographic optical elements (HOEs), and spatial light modulators (SLM). In the example of FIG. 6, the optical elements 664 are collimators. The output beams 660 can be guided via the beam scanner 316 to a cornea, such as the cornea 346 of FIG. 3, as generally described relative to FIGS. 1A-B, 2, and 3.

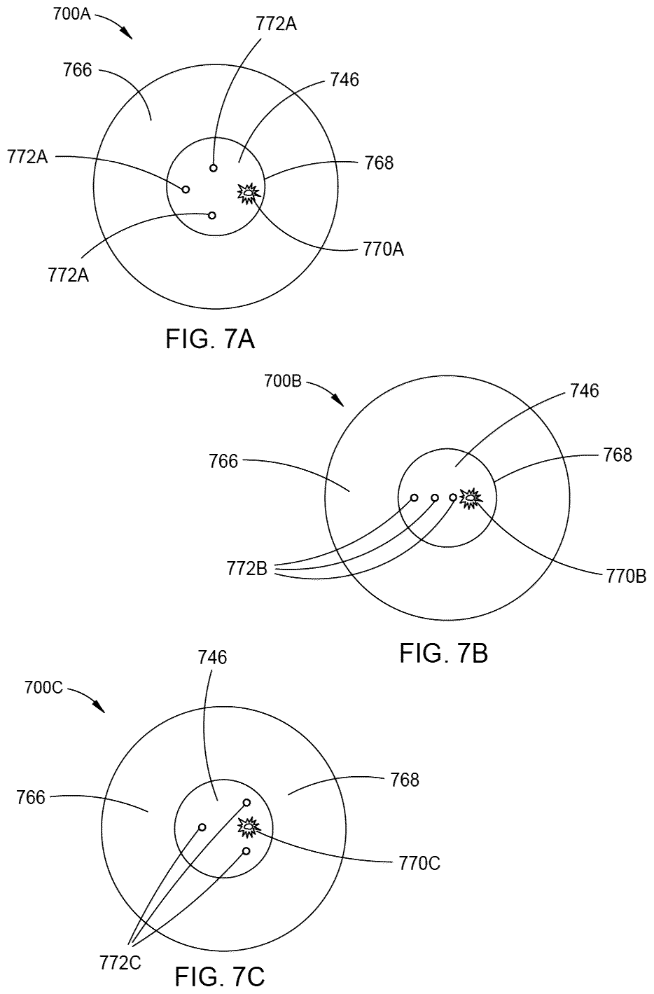

FIGS. 7A-C illustrate example measurement patterns for an eye. The eye is shown with cornea 746, conjunctiva 766, and limbus 768. FIG. 7A shows a measurement pattern 700A. In the measurement pattern 700A, a stimulus is applied, for example, via the stimulation device 352 of FIG. 3, at a stimulation location 770A on the cornea 746. In response to the stimulus, imaging light beams are emitted, at approximately the same time (i.e., simultaneously), to the measurement locations 772A, so that corneal responses may be measured.

FIG. 7B illustrates an example measurement pattern 700B. In the measurement pattern 700B, a stimulus is applied, for example, via the stimulation device 352 of FIG. 3, at a stimulation location 770B on the cornea 746. In response to the stimulus, imaging light beams are emitted, at approximately the same time (i.e., simultaneously), to the measurement locations 772B, so that corneal responses may be measured.

FIG. 7C illustrates an example measurement pattern 700C. In the measurement pattern 700C, a stimulus is applied, for example, via the stimulation device 352 of FIG. 3, at a stimulation location 770C on the cornea 746. In response to the stimulus, imaging light beams are emitted, at approximately the same time (i.e., simultaneously), to the measurement locations 772C, so that corneal responses may be measured.

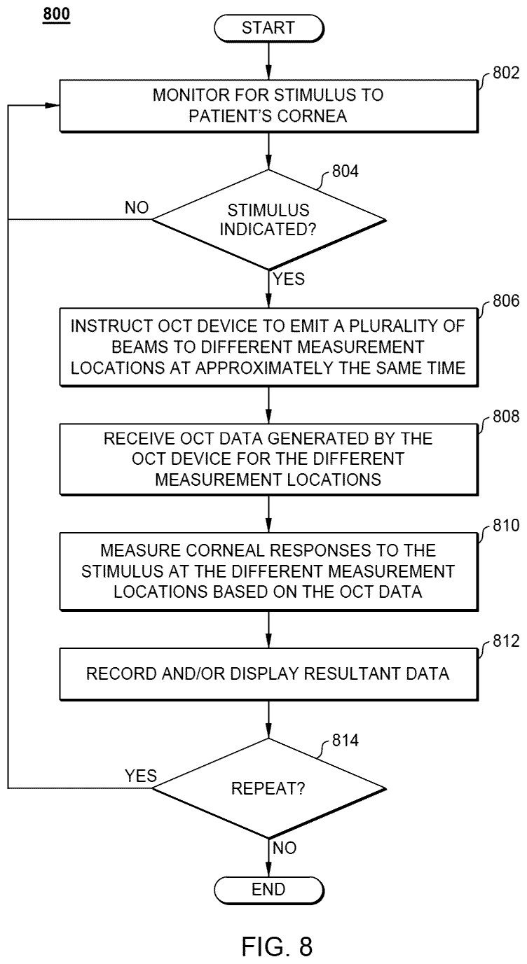

FIG. 8 illustrates an example of a process 800 for measuring corneal biomechanics in real-time at multiple corneal locations. In certain embodiments, the process 800 can be implemented by any system that can process OCT data. Although any number of systems, in whole or in part, can implement the process 800, to simplify discussion, the process 800 will be described in relation to example components of the ophthalmic diagnostics system 10 of FIGS. 1A-B and 2 and the ophthalmic diagnostics system 310 of FIG. 3.

At block 802, the computer 30 monitors for an indication that a stimulus has been applied to a patient's cornea, such as the cornea 346 of FIG. 3. The indication can be, for example, an automatic detection that a stimulus has been applied, a user-supplied input that a stimulus has been applied, a synchronization signal generated by the computer, an expiration of a synchronization timer for applying a stimulus, combinations of the foregoing, and/or the like. In various embodiments, indications of a stimulus may include, or be accompanied by, an indication of a location of the stimulus. The stimulus can be applied, for example, via the stimulation device 352 of FIG. 3, to stimulation locations such as the stimulation locations 770A, 770B, and 770C of FIGS. 7A, 7B, and 7C, respectively.

At decision block 804, the computer 30 determines whether an indication of a stimulus applied to the patient's eye has been received. If no indication of a stimulus has been received, the process 800 returns to the block 802 and continues as described previously. Otherwise, if it is determined at the decision block 804 that an indication of a stimulus has been received, the process 800 proceeds to block 806.

At block 806, the computer 30 instructs the OCT device 15 to emit a plurality of imaging light beams, at approximately the same time (i.e., simultaneously), to a plurality of desired measurement locations on the patient's cornea. In an example, the desired measurement locations can correspond to the measurement locations 772A, 772B, or 772C of FIGS. 7A, 7B, and 7C, respectively. The instruction to the OCT device 15 can cause the OCT device 15 to generate and emit a plurality of imaging light beams in any of the ways described above relative to FIGS. 1A-B and 2-6, and provide OCT data based thereon. At block 808, the computer 30 receives, from the OCT device 15, OCT data generated by the OCT device 15 for each of the plurality of measurement locations.

At block 810, the computer measures corneal responses to the stimulus at the plurality of measurement locations based on the OCT data. For example, the computer 30 can measure a propagation speed of a shear wave along or across the patient's cornea, based on the measurement locations. In certain embodiments, the computer 30 can quantify tissue stiffness (e.g., Young's modulus), for example, based on the propagation speed of the shear wave. For example, if viscosity can be neglected, Young's modulus (E) can be related to the propagation speed of the shear wave, or shear wave speed (S_{c}), based on Equation 1 below, where ρ is the material density and mv is Poisson's ratio. In general, tissue stiffness is proportional to Young's modulus and a higher Young's modulus corresponds to greater (lengthwise) stiffness.

$$E = 2 * ρ * ( 1 + mv ) * {S}_{c} \qquad Equation 1$$

At block 812, the computer 30 stores and/or displays resultant data from the blocks 808 and 810, such as OCT data and resultant corneal biomechanics. In various embodiments, the OCT data and/or resultant corneal biomechanics can be stored in relation to the patient in the memory 32 or other storage. In addition, or alternatively, the OCT data and/or resultant biomechanics can be displayed to a user or operator of the ophthalmic diagnostics system 10.

At decision block 814, the computer 30 determines whether to collect additional corneal biomechanics responsive to further stimulation of the patient's eye. If it is determined at the decision block 814 to collect additional corneal biomechanics, the process 800 returns to the block 802 and executes as described previously. Otherwise, the process 800 ends.

In various embodiments, diagnostic systems such as the example diagnostic systems described herein can have various advantages. For example, such a diagnostics system that measures biomechanical properties of diseased or healthy corneas allows for new metrics to be included in treatment planning algorithms to increase predictability and surgeon confidence. For example, corneal biomechanics such as those described herein can help evaluate therapeutic interventions in comparison with cross-linking, as well as help evaluate collagen degradation. Further, in some embodiments, corneal biomechanics is highly correlated with myopia, thereby affecting orthokeratology (Ortho-K) success (e.g., myopia reduction). High myopia may increase the risk of glaucoma. Thus, in certain embodiments, corneal biomechanics such as those described herein can help predict or identify risk of myopia and/or glaucoma.

Additionally, or alternatively, in various embodiments, diagnostic systems such as the example diagnostic systems described herein can help identify patients at risk of post-LASIK complications. Typically, refractive surgery planning uses a population-based average of corneal biomechanics. Statistically, approximately one percent of LASIK patients experience ectasia. In various embodiments, individualized corneal biomechanics as described herein may improve the ability to predict the risks of surgical intervention, such as post-LASIK ectasia.

Additionally, or alternatively, the ability to produce measurements of corneal biomechanics and apply them to treatment algorithms can support more accurate estimation, prediction, and/or establishment of cataract outcomes from patient-specific surgically induced astigmatism (SIA), Limbal Relaxing Incision (LRI) outcomes from patient-specific calculations, treatment decisions for corneal refractive power, Ortho-K outcomes, treatment and/or diagnosis of dry eye, and/or the like. For example, SIA can range from 0 to 1.5D, which can be a meaningful source of refractive surprises. Corneal biomechanics such as those described herein can enable better prediction of a patient-specific SIA for cataract surgery and increase the accuracy of LRIs.

The above-disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description.

What is claimed is:

1. A system for measuring biomechanics in real-time at multiple eye-tissue locations, the system comprising:
an optical coherence tomography (OCT) device; and
a computer communicably coupled to the OCT device, wherein the computer is operable to:
receive an indication of a stimulus applied to eye tissue of a patient;
instruct the OCT device to emit a plurality of beams, at approximately the same time, to a plurality of measurement locations on the eye tissue in response to the received indication;
receive, from the OCT device, OCT data for each of the plurality of measurement locations; and
measure tissue responses to the stimulus at the plurality of measurement locations based on the OCT data.

2. The system of claim 1, the OCT device comprising an OCT engine that generates the plurality of beams at approximately the same time in response to the instruction.

3. The system of claim 2, the OCT device further comprising a beam scanner, an optical element, and a focusing objective, wherein:
the beam scanner guides the generated plurality of beams to a first plurality of locations on the optical element;
the optical element directs the guided plurality of beams toward a second plurality of locations on the focusing objective based on the first plurality of locations; and
the focusing objective focuses the directed plurality of beams on the plurality of measurement locations on the eye tissue.

4. The system of claim 3, wherein the optical element is a dichroic mirror.

5. The system of claim 2, the OCT engine comprising a beam splitter and first and second optical elements, wherein:
the beam splitter splits a source beam into a first beam to the first optical element and a second beam to the second optical element;
the first and second optical elements direct the first and second beams to a beam scanner; and
the plurality of beams generated by the OCT engine comprise the directed first and second beams.

6. The system of claim 2, the OCT engine comprising a fiber beam splitter and first and second optical elements, wherein:
the fiber beam splitter splits a source beam into a first beam to the first optical element and a second beam to the second optical element;
the first and second optical elements direct the first and second beams to a beam scanner; and
the plurality of beams generated by the OCT engine comprise the directed first and second beams.

7. The system of claim 2, the OCT engine comprising a plurality of beam splitters including first, second and third beam splitters and a plurality of optical elements including first, second, third and fourth optical elements, wherein:
the first beam splitter splits a source beam into a first intermediate beam to the second beam splitter and a second intermediate beam to the third beam splitter;
the second beam splitter splits the first intermediate beam into a first output beam to the first optical element and a second output beam to the second optical element;
the third beam splitter splits the second intermediate beam into a third output beam to the third optical element and a fourth output beam to the fourth optical element;
the first, second, third, and fourth optical elements direct the first, second, third, and fourth output beams to a beam scanner; and
the plurality of beams generated by the OCT engine comprise the directed first, second, third, and fourth output beams.

8. The system of claim 1, wherein the computer is operable to at least one of record or display data resultant from the measured tissue responses.

9. The system of claim 1, wherein the measurement comprises measurement of a propagation speed of a shear wave across at least a portion of the eye tissue based on the OCT data.

10. The system of claim 9, wherein the measurement comprises a quantification of tissue stiffness based on the propagation speed of the shear wave.

11. The system of claim 1, wherein the eye tissue comprises a cornea of the patient.

12. A method of measuring biomechanics in real-time at multiple eye-tissue locations, the method comprising, by a computer in communication with an optical coherence tomography (OCT) device:
receiving an indication of a stimulus applied to eye tissue of a patient;
instructing the OCT device to emit a plurality of beams, at approximately the same time, to a plurality of measurement locations on the eye tissue in response to the received indication;
receiving, from the OCT device, OCT data for each of the plurality of measurement locations; and
measuring tissue responses to the stimulus at the plurality of measurement locations based on the OCT data.

13. The method of claim 12, comprising at least one of recording or displaying data resultant from the measured tissue responses.

14. The method of claim 12, wherein the measuring tissue responses comprises measuring a propagation speed of a shear wave across at least a portion of the eye tissue based on the OCT data.

15. The method of claim 14, wherein the measuring comprises quantifying tissue stiffness based on the propagation speed of the shear wave.

16. The method of claim 12, further comprising the OCT device generating the plurality of beams at approximately the same time in response to the instructing.

17. The method of claim 16, further comprising:
guiding, via a beam scanner, the generated plurality of beams to a first plurality of locations on an optical element;
directing, via the optical element, the guided plurality of beams toward a second plurality of locations on a focusing objective based on the first plurality of locations; and
focusing, via the focusing objective, the directed plurality of beams on the plurality of measurement locations on the eye tissue.

18. The method of claim 16, the generating the plurality of beams comprising:
splitting a source beam into a first beam to a first optical element and a second beam to a second optical element; and
directing, via the first and second optical elements, the first and second beams to a beam scanner, wherein the generated plurality of beams comprise the directed first and second beams.

19. The method of claim 16, the generating the plurality of beams comprising
splitting a source beam into a first intermediate beam and a second intermediate beam;
splitting the first intermediate beam into a first output beam to a first optical element and a second output beam to a second optical element;
splitting the second intermediate beam into a third output beam to a third optical element and a fourth output beam to a fourth optical element; and
directing, via the first, second, third, and fourth optical elements, the first, second, third, and fourth output beams to a beam scanner, wherein the generated plurality of beams comprise the directed first, second, third, and fourth output beams.

20. A computer-program product comprising a non-transitory computer-usable medium having computer-readable program code embodied therein, the computer-readable program code adapted to be executed to implement a method comprising:
receiving an indication of a stimulus applied to eye tissue of a patient;
instructing an optical coherence tomography (OCT) device to emit a plurality of beams, at approximately the same time, to a plurality of measurement locations on the eye tissue in response to the received indication;
receiving, from the OCT device, OCT data for each of the plurality of measurement locations; and
measuring tissue responses to the stimulus at the plurality of measurement locations based on the OCT data.
